(19) United States
(12) Patent Application Publication
(10) Pub. No.: US 2022/0399110 A1
(43) Pub. Date: Dec. 15, 2022

(54) MEDICAL INFORMATION PROCESSING APPARATUS AND MEDICAL INFORMATION PROCESSING SYSTEM

(71) Applicant: Canon Medical Systems Corporation, Otawara-shi (JP)

(72) Inventors: Yusuke KANO, Nasushiobara (JP); Anri YAMAZAKI, Nasushiobara (JP)

(73) Assignee: Canon Medical Systems Corporation, Otawara-shi (JP)

(21) Appl. No.: 17/805,303

(22) Filed: Jun. 3, 2022

(30) Foreign Application Priority Data
Jun. 15, 2021 (JP) 2021-099384

(51) Int. Cl. G16H 40/67; G16H 10/60; G16H 80/00; A61B 5/00

(52) U.S. Cl. CPC G16H 40/67; G16H 10/60; G16H 80/00; A61B 5/0022

(57) ABSTRACT

According to one embodiment, a medical information processing apparatus includes processing circuitry.

The processing circuitry acquires a first numerical value and a second numerical value, the first numerical value corresponding to a user's judgement based on an observed confounding factor, the second numerical value corresponding to the user's judgement based on the observed confounding factor and support information that supports the user's judgement. The processing circuitry extracts a difference between the first and second numerical values. The processing circuitry calculates a degree of influence of an unobserved confounding factor on the user's judgement based on the difference and the observed confounding factor.

### CROSS-REFERENCE TO RELATED APPLICATIONS

This application is based upon and claims the benefit of priority from Japanese Patent Application No. 2021-099384, filed Jun. 15, 2021, the entire contents of which are incorporated herein by reference.

### FIELD

Embodiments described herein relate generally to a medical information processing apparatus and a medical information processing system.

### BACKGROUND

Causal inference is a method for estimating a causal effect of intervention or exposure on outcomes from data and is used in a wide range of fields such as medical services, economics, politics, and marketing. In recent years, many methods for estimating an individual causal effect from data using machine learning (e.g., TARNet, Causal Forest, CMGP, GANITE, X-learner) have been proposed. In such causal inference using machine learning, it is necessary to identify all confounding factors that affect a causal relationship in order to properly estimate the causal effect.

However, in order to identify the confounding factors, human expertise (domain knowledge) in the target field is theoretically indispensable, and it is generally difficult to identify all confounding factors. Furthermore, since there is no means to strictly verify from the data whether or not domain knowledge and a causal inference result are correct, there is room for unobserved confounding factors to be present. Methods for estimating a causal effect in the presence of unobserved confounding factors include, for example, a randomized controlled trial (RCT), a regression discontinuity design (RDD), an instrumental variable (IV), and a front door criteria, but these are not realistic due to strict conditions. In addition, many of the methods of causal inference by machine learning proposed in recent years assume that there are no unobserved confounding factors, but the validity of this assumption is disregarded in actual analysis. Therefore, in order to appropriately estimate a causal effect in causal inference using machine learning, it is desirable to quantify a degree of influence of unobserved confounding factors.

### BRIEF DESCRIPTION OF THE DRAWINGS

FIG. 1 is a configuration example of a medical information processing system according to an embodiment.

FIG. 2 is a configuration example of a medical information processing apparatus according to the embodiment.

FIG. 3 is an operation example of the medical information processing apparatus.

FIG. 4 is an example of a method for collecting a data set for causal inference.

FIG. 5 is an example of a data set for causal inference.

FIG. 6 is an example of a method for training a parameter of a prediction function of a propensity score.

FIG. 7 is an example of a degree of influence of each confounding factor on support information.

### DETAILED DESCRIPTION

In general, according to one embodiment, a medical information processing apparatus includes processing circuitry.

The processing circuitry acquires a first numerical value corresponding to a result judged by a user based on an observed confounding factor or based on the observed confounding factor and an unobserved confounding factor. The processing circuitry acquires a second numerical value corresponding to a result judged by the user based on the observed confounding factor and first support information that supports a judgment of the user or based on the observed confounding factor, the unobserved confounding factor, and the first support information. The processing circuitry extracts a first difference between the first numerical value and the second numerical value. The processing circuitry calculates a degree of influence of the unobserved confounding factor on the judgment of the user based on the first difference and the observed confounding factor.

Hereinafter, a medical information processing apparatus and a medical information processing system according to an embodiment will be described with reference to the drawings. In the following embodiment, portions assigned the same reference sign perform the same operation, and redundant explanations will be omitted as appropriate.

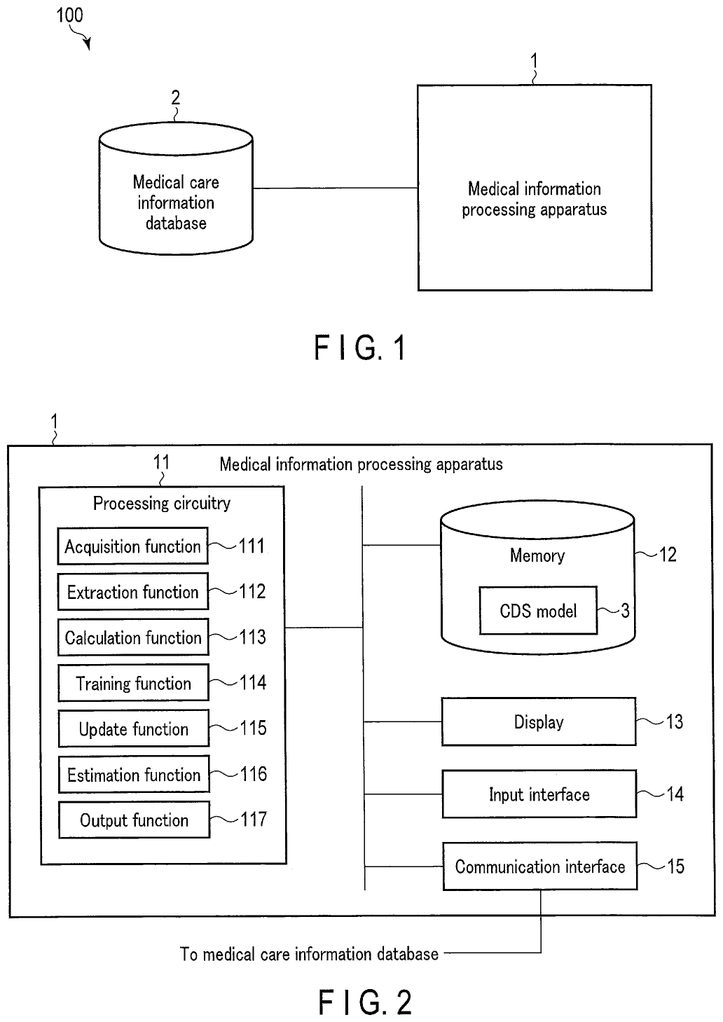

FIG. 1 is a configuration example of a medical information processing system 100 according to the embodiment.

The medical information processing system 100 includes a medical information processing apparatus 1 and a medical care information database 2. In the medical information processing system 100, the medical information processing apparatus 1 and the medical care information database 2 are connected to each other to enable communications therebetween. The medical information processing system 100 may be, for example, an in-hospital network (LAN) constructed in a specific medical institution, or a wide area network (WAN) constructed across a plurality of medical institutions via a network. That is, the medical information processing system 100 may be a network of any scale as long as the above communication path is constructed.

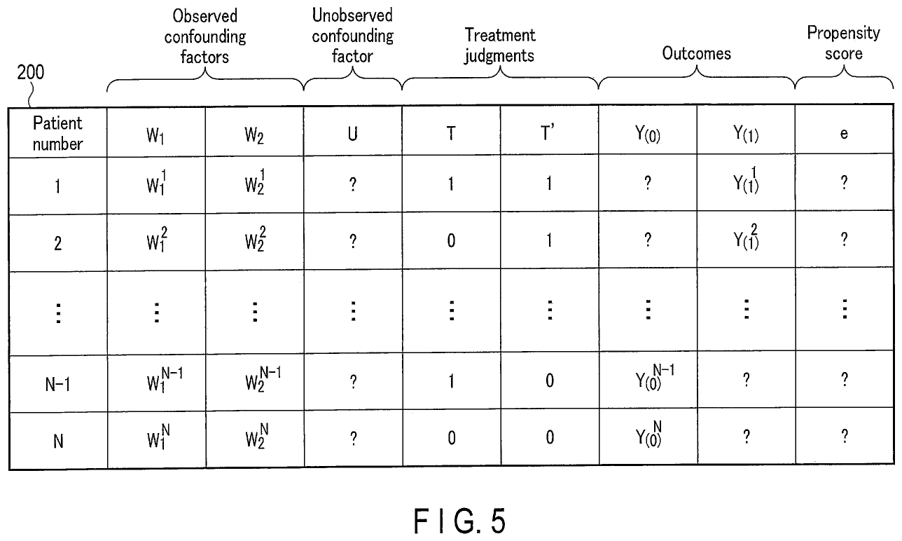

The medical information processing apparatus 1 is a computer adapted to process various information on medical services. Specifically, the medical information processing apparatus 1 acquires a data set 200 for causal inference (to be described later in FIG. 5) from the medical care information database 2 and performs various processing to quantify a degree of influence of an unobserved confounding factor. The medical information processing apparatus 1 may be a workstation capable of performing high-speed processing.

The medical care information database 2 stores various medical care information for each patient. The medical care information includes, for example, basic information (patient number, age, gender, date of birth, etc.), personal information (height, weight, blood type, medical history, presence or absence of illness, lifestyle habits (exercise, smoking, diet, drinking, stress, sleep), etc.), and disease information (disease name, disease stage, frailty score, treatment method performed (surgery or medication), prognosis after treatment, etc.). Furthermore, the medical care information includes medical images taken by various medical image diagnostic devices (a computer radiography (CR) device, a computed tomography (CT) device, a magnetic resonance imaging (MRI) device, an ultrasound (UL) device, a radio isotope (RI) device, an endoscope device, etc.). In the present embodiment, the medical care information database 2 includes a data set 200 for causal inference. The medical care information database 2 may be stored in the medical information processing apparatus 1.

FIG. 2 is a configuration example of the medical information processing apparatus 1 according to the embodiment.

The medical information processing apparatus 1 includes processing circuitry 11, a memory 12, a display 13, an input interface 14, and a communication interface 15. The configurations are connected to one another via a bus which is a common signal transmission path to enable communications therebetween. Each configuration need not be realized by an individual piece of hardware. For example, at least two of the configurations may be realized by a single piece of hardware.

The processing circuitry 11 controls the medical information processing apparatus 1 to execute various operations. The processing circuitry 11 includes, as hardware, a processor such as a central processing unit (CPU), a micro processing unit (MPU), or a graphics processing unit (GPU). By executing programs developed in the memory 12 via the processor, the processing circuitry 11 realizes functions (e.g., an acquisition function 111, an extraction function 112, a calculation function 113, a training function 114, an update function 115, an estimation function 116, and an output function 117) respectively corresponding to the programs. Each function may be realized by the processing circuitry 11 in which a plurality of processors are combined.

The acquisition function 111 acquires a first numerical value corresponding to a result judged by a user based on an observed confounding factor. Further, the acquisition function 111 acquires a second numerical value corresponding to a result judged by the user based on the observed confounding factor and first support information that supports the user's judgment.

The extraction function 112 extracts a first difference between the first numerical value and the second numerical value. The extraction function 112 also extracts a second difference between a first propensity score and a second propensity score. The first propensity score and the second propensity score are a predicted value of the first numerical value and a predicted value of the second numerical value, respectively.

The calculation function 113 calculates a degree of influence of an unobserved confounding factor on the user's judgment based on the first difference and the observed confounding factor.

The training function 114 trains a first parameter of a first function and a second parameter of a second function so as to minimize a prediction residual between the first difference and the second difference.

The update function 115 updates a model that outputs first support information using the degree of influence of the unobserved confounding factor.

The estimation function 116 estimates a causal effect of the user's judgment on an outcome based on the degree of influence of the unobserved confounding factor.

The output function 117 outputs second support information that supports the user's judgment based on the causal effect. The output function 117 also outputs a ratio of the degree of influence of the unobserved confounding factor in the second support information. Further, the output function 117 outputs a candidate for an unobserved confounding factor that affects the second support information.

The memory 12 stores information of data and programs, etc. used by the processing circuitry 11. The memory 12 has a semiconductor memory device such as a random access memory (RAM) as hardware. The memory 12 may be a driving device that reads and writes information to and from external storage devices, such as a magnetic disk (a floppy (registered trademark) disk, a hard disk), a magneto-optical disk (MO), an optical disk (a CD, a DVD, a Blu-ray (registered trademark)), a flash memory (a USB flash memory, a memory card, and an SSD), and a magnetic tape. A storage region of the memory 12 may be in an inner portion of the medical information processing apparatus 1 or in an external storage device. In the present embodiment, the memory 12 stores a first function that outputs a first propensity score, which is a predicted value of a first numerical value, with an observed confounding factor as an input, and a second function that outputs a second propensity score, which is a predicted value of a second numerical value, with an observed confounding factor as an input. Furthermore, the memory 12 stores a clinical decision support (CDS) model 3. The memory 12 is an example of a storage unit.

The CDS model 3 supports clinical decision-making of a user who uses the medical information processing apparatus 1. Users include, for example, medical staff, such as a doctor and a nurse who treat a patient. In the present embodiment, it is assumed that the CDS model 3, with a plurality of types of medical care information regarding a patient as inputs, outputs support information that supports a judgment of a doctor who treats that patient. The configuration is not limited thereto; the CDS model 3 may output information (raw data, a prediction, a recommendation, etc.) that can change the doctor's judgment. The CDS model 3 is implemented by a machine learning model such as a neural network.

The display 13 displays data generated by the processing circuitry 11, data stored in the memory 12, data output by the CDS model 3, etc. As the display 13, any display including, for example, a cathode ray tube (CRT) display, a liquid crystal display (LCD), a plasma display, an organic electro-luminescence display (OELD), and a tablet terminal, can be used.

The input interface 14 receives an input from a user who uses the medical information processing apparatus 1, and converts the received input into an electric signal and outputs the electric signal to the processing circuitry 11. As the input interface 14, any operation component including, for example, a mouse, a keyboard, a trackball, a switch, a button, a joystick, a touch pad, and a touch panel display, can be used. The input interface 14 may be a device that receives an input from an external input device that is separate from the medical information processing apparatus 1, converts the received input into an electric signal, and outputs the electric signal to the processing circuitry 11.

The communication interface 15 communicates various data between the medical information processing apparatus 1 and the medical care information database 2. As communication standards, for example, DICOM (Digital Imaging and Communications in Medicine) can be used for communication related to medical image information, and HL7 (Health Level 7) can be used for communication related to medical character information.

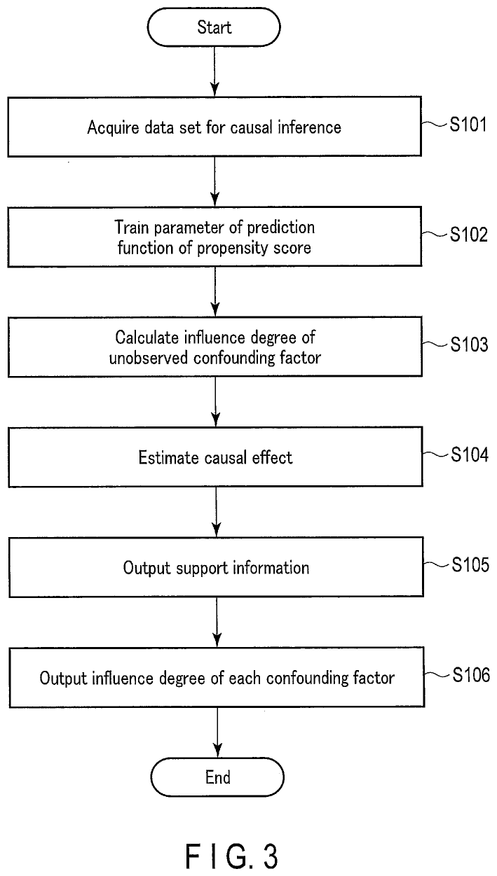

FIG. 3 is an operation example of the medical information processing apparatus 1.

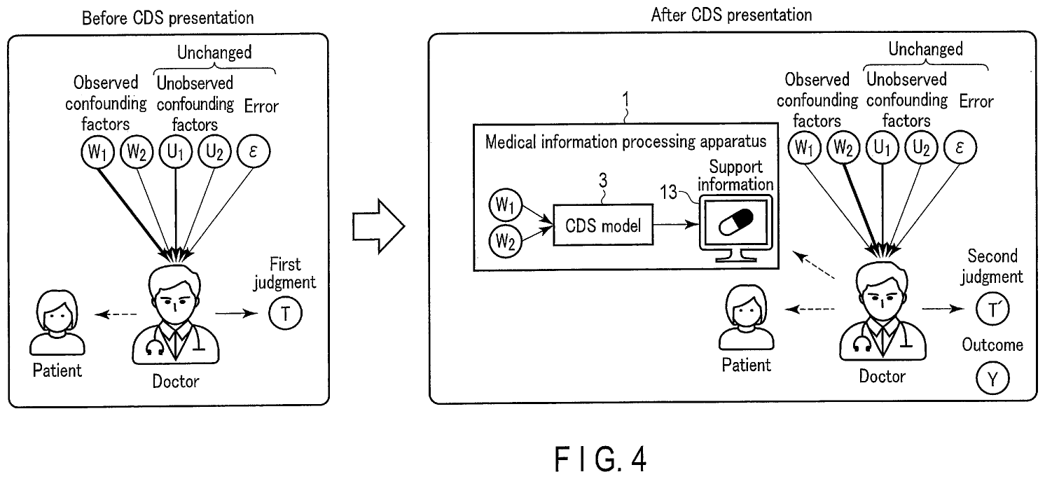

In step S101, the medical information processing apparatus 1 acquires the data set 200 for causal inference by the acquisition function 111. Specifically, the medical information processing apparatus 1 acquires the data set 200 for causal inference by accessing the medical care information database 2 via the communication interface 15. The data set 200 includes a first numerical value corresponding to a result judged by a user based on an observed confounding factor and a second numerical value corresponding to a result judged by the user based on the observed confounding factor and first support information that supports the user's judgment. The data set 200 may be stored in the medical care information database 2 in advance, or may be newly collected by the medical information processing apparatus 1 according to a method shown in FIG. 4.

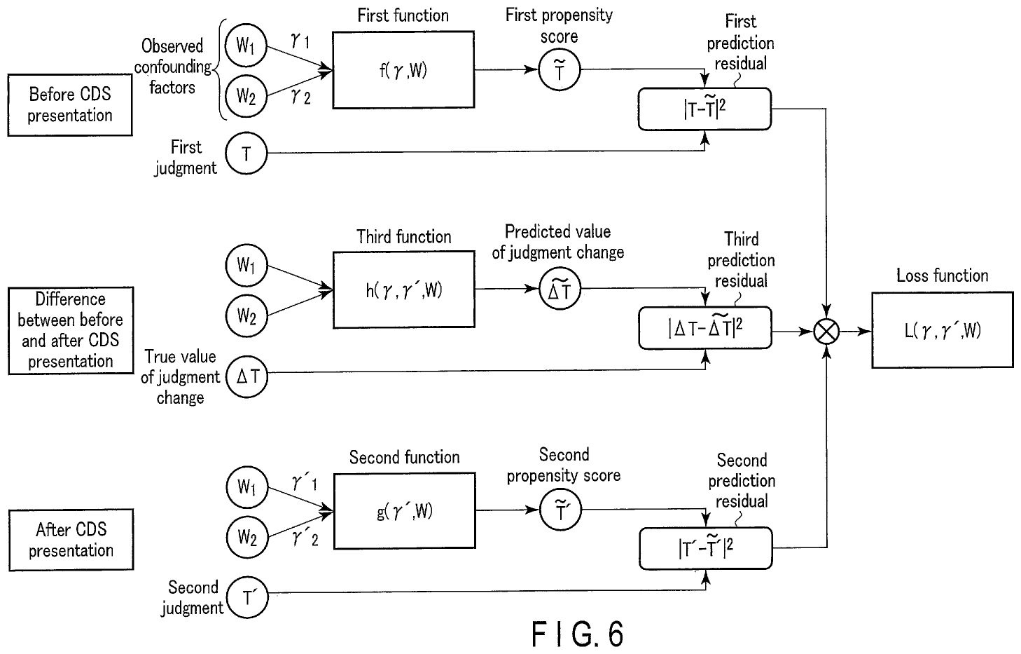

In step S102, the medical information processing apparatus 1 trains a parameter of a prediction function of a propensity score using the training function 114. Specifically, by using the acquired data set 200, the medical information processing apparatus 1 trains a first parameter of a first function that predicts a first propensity score, which is a predicted value of the first numerical value, and a second parameter of a second function that predicts a second propensity score, which is a predicted value of the second numerical value. Details of parameter training will be described later in FIG. 6.

In step S103, the medical information processing apparatus 1 calculates a degree of influence of an unobserved confounding factor using the calculation function 113. Specifically, the medical information processing apparatus 1 calculates a difference between the first numerical value and the first propensity score predicted using the trained first parameter, or a difference between the second numerical value and the second propensity score predicted using the trained second parameter, as the degree of influence of the unobserved confounding factor.

In step S104, the medical information processing apparatus 1 estimates a causal effect using the estimation function 116. Specifically, the medical information processing apparatus 1 estimates the causal effect of the user's judgment on an outcome based on the calculated degree of influence of the unobserved confounding factor. Further, using the update function 115, the medical information processing apparatus 1 may update a model (CDS model 3) that outputs the first support information that supports the user's judgment using the calculated degree of influence of the unobserved confounding factor.

In step S105, the medical information processing apparatus 1 outputs support information using the output function 117. Specifically, the medical information processing apparatus 1 or the CDS model 3 outputs second support information that supports the user's judgment based on the estimated causal effect.

In step S106, the medical information processing apparatus 1 outputs a degree of influence of each confounding factor using the output function 117. Specifically, the medical information processing apparatus 1 outputs a ratio of the degree of influence of the unobserved confounding factor in the second support information. Further, the medical information processing apparatus 1 may output a candidate for an unobserved confounding factor that affects the second support information, using the output function 117.

FIG. 4 is an example of a method for collecting the data set 200 for causal inference.

Hereinafter, as an example of causal inference, we focus on a causal relationship between a doctor's judgment on a patient's treatment method (also referred to as a treatment judgment) and a lifespan of that patient when the patient is treated based on that judgment. In this causal relationship, the doctor's judgment corresponds to intervention T (treatment), and the lifespan of the patient due to the intervention T corresponds to an outcome Y. At this time, it is considered that there are a plurality of confounding factors that distort the causal relationship between the intervention T and the outcome Y. The plurality of confounding factors are divided into a confounding factor that is objectively clear and observed for reasons such as data being obtained (also referred to as an observed confounding factor W), and a confounding factor whose data is not obtained and which is not objectively clear and not observed as well as a factor that has data obtained but is not recognized as a confounding factor (also referred to as an unobserved confounding factor U). These confounding factors affect the doctor's judgment T with different degrees of influence, and also affect the patient's lifespan Y. In the present embodiment, it is assumed that the doctor explicitly considers the observed confounding factor W and implicitly considers the unobserved confounding factor U to make the judgment T. A degree of influence of each confounding factor on the doctor's judgment T is illustrated by arrows having different thicknesses.

In order to collect the data set 200 for causal inference, in this method, the doctor judges a treatment method for the patient before and after the CDS model 3 presents support information. Here, it is assumed that the degrees of influence of the unobserved confounding factor U on the doctor's judgment and an error ε in judgment are unchanged or constant before and after the presentation of the support information. Conversely, the degree of influence of the observed confounding factor W on the doctor's judgment changes before and after the presentation of the support information.

First, before the presentation of the support information (before the CDS presentation), the doctor makes a judgment based on the observed confounding factor W and the unobserved confounding factor U. For example, a case is assumed in which the observed confounding factors W are age W₁ and disease stage W₂, and the unobserved confounding factors U are frailty U₁ and gender U₂. Considering the patient's age W₁ and disease stage W₂, the doctor makes a first judgment T regarding a treatment method for that patient. The age W₁ is a quantitative variable that can take any numerical value, and the disease stage W₂ is a qualitative variable having a plurality of categories. Specifically, the doctor makes the first judgment T by placing more importance on the patient's age W₁ than on the disease stage W₂. At this time, it is assumed that the doctor makes the first judgment T by further considering the patient's frailty U₁ and gender U₂, which are the unobserved confounding factors U, implicitly. Specifically, the degree of influence of the frailty U₁ is slightly higher than the degree of influence of the gender U₂.

The first judgment T is a qualitative variable having a plurality of categories. In the present embodiment, the first judgment T is a binary variable having two categories, “surgery” or “medication”. Specifically, “surgery” is expressed as “T=1” and “medication” is expressed as “T=0”, using dummy variables. Of course, the first judgment T may be a multi-valued variable having three or more categories. That is, the first judgment T may be expressed by an N-dimensional one-hot vector corresponding to the number N (N is a natural number) of each category. The first judgment T is stored in the medical care information database 2.

Subsequently, the medical information processing apparatus 1 displays the support information on the display 13 via the CDS model 3. Specifically, the medical information processing apparatus 1 inputs the age W₁ and the disease stage W₂, which are the observed confounding factors W before the CDS presentation, to the CDS model 3. The CDS model 3 outputs the support information that supports the doctor's judgment based on the input patient's age W₁ and disease stage W₂. For example, the CDS model 3 outputs a treatment method (also referred to as a recommended treatment) recommended for the patient as the support information. It is assumed that the recommended treatment is not included in the observed confounding factors W because it affects a judgment T′ of the doctor after the CDS presentation but does not affect the patient's lifespan Y.

The configuration is not limited thereto, and the CDS model 3 may output support information that also affects the patient's lifespan Y. For example, with the patient's age W₁ and disease stage W₂ as inputs, the CDS model 3 may output that patient's frailty score W₃. The frailty score W₃ is included in the observed confounding factors W because it affects the doctor's judgment T′ after the CDS presentation as well as the patient's lifespan Y. The doctor reconsiders the judgment of the treatment method for the patient by confirming the support information displayed on the display 13. The medical information processing apparatus 1 may present to the doctor raw data of the observed confounding factor W that should be referred to for the treatment judgment as the support information. That is, the support information may be any factor that can change the doctor's treatment judgment.

The support information may be a value composed of or calculated from, among a plurality of observed confounding factors, all or some of the observed confounding factors. As an example, when a plurality of observed confounding factors W1, W2, W3, and W4 are present, the support information may be a value calculated from the observed confounding factors W1 and W2, which are some of the observed confounding factors.

Finally, after the support information is presented (after the CDS presentation), the doctor makes a judgment based on the observed confounding factors W, the support information, and the unobserved confounding factors U. For example, considering the patient's age W₁, disease stage W₂, and the recommended treatment presented by the CDS model 3, the doctor makes the second judgment T′ about the treatment method for that patient. Here, the doctor makes the second judgment T′ by placing more importance on the patient's disease stage W₂ than on the age W₁. As described above, since it is assumed that the degrees of influence of the unobserved confounding factors U and the error € are unchanged in the first judgment T and the second judgment T′, a change in doctor's judgment from the first judgment T to the second judgment T′ can be deemed to be due to a change in degree of influence of the observed confounding factors W.

The second judgment T′ is a qualitative variable having a plurality of categories. In the present embodiment, the second judgment T′ is a binary variable having two categories, “surgery” or “medication”. Specifically, “surgery” is expressed as “T′=1” and “medication” is expressed as “T′=0”, using dummy variables. Of course, the second judgment T′ may be a multi-valued variable having three or more categories. That is, the second judgment T′ may be expressed by an N-dimensional one-hot vector corresponding to the number N (N is a natural number) of each category. In other words, definitions of the first judgment T and the second judgment T′ are the same. The second judgment T′ is stored in the medical care information database 2.

Further, the lifespan Y of the patient, which is a result of the treatment performed on that patient based on the second judgment T′, is stored in the medical care information database 2. In the present embodiment, the lifespan Y is a quantitative variable that can take any numerical value. The lifespan Y is divided into a lifespan Y₍₁₎ in the case where the second judgment T′ is “surgery” (T′=1) and a lifespan Y₍₀₎ in the case where the second judgment T′ is “medication” (T′=0). For one patient, either Y₍₀₎ or Y₍₀₎ is observed, but the other is not, so the unobserved outcome Y₍₁₎ or Y₍₀₎ is also referred to as a potential outcome.

By the above judgment flow, data in which values of the observed confounding factors W₁ and W₂, the first judgment T, the second judgment T′, and the outcome Y₍₁₎ or Y₍₀₎ are associated for one patient is stored in the medical care information database 2. By repeating the same flow for each of a plurality of patients, a data set 200 for causal inference in which the above respective values are associated for each patient is collected. As described above, it can be said that the data set 200 is not pure observation data because in this method, an operation similar to an experiment in which the user makes a judgment twice is performed.

FIG. 5 is an example of the data set 200 for causal inference.

In the data set 200, values of observed confounding factors W₁ and W₂, an unobserved confounding factor U, treatment judgments T and T′, and an outcome Y₍₀₎ or Y₍₁₎ are associated for each of N patients (N is a natural number) and stored. For each patient, a value of each of the unobserved confounding factor U and the potential outcome Y₍₀₎ or Y₍₁₎ is unknown, so cells with unknown values are indicated by “?”. The unobserved confounding factors U₁ and U₂ are simply shown as “U”.

For example, for a patient represented by patient number “1”, the respective values are W₁=W₁¹, W₂=W₂¹, T=1, T′=1, Y₍₁₎=Y₍₁₎¹. In other words, the patient's age W₁ is W₁¹, and disease stage W₂ is W₂¹. That is, according to the data set 200, a case can be grasped in which the doctor selects “surgery” as the treatment judgment T before the CDS presentation for the patient, selects “surgery” as the treatment judgment T′ after the CDS presentation, and as a result of performing “surgery” on the patient based on the latter treatment judgment T′, the patient survived only for a period of Y₍₁₎¹. That is, it can be seen that the doctor's judgment did not change before and after the CDS presentation in this case.

Similarly, for a patient represented by patient number “2”, the respective values are W₁=W₁², W₂=W₂², T=0, T′=1, Y₍₁₎=Y₍₁₎². In other words, the patient's age W₁ is W₁², and disease stage W₂ is W₂². That is, according to the data set 200, a case can be grasped in which the doctor selects “medication” as the treatment judgment T before the CDS presentation for the patient, selects “surgery” as the treatment judgment T′ after the CDS presentation, and as a result of performing “surgery” on the patient based on the latter treatment judgment T′, the patient survived only for a period of Y₍₁₎². That is, it can be seen that the doctor's judgment changed before and after the CDS presentation in this case.

Next, the medical information processing apparatus 1 performs training based on the data set 200 for causal inference so as to estimate a causal effect Y₍₁₎−Y₍₀₎ of the doctor's treatment judgment T on the patient's lifespan Y. Here, it is assumed that a prediction formula of the outcome Y for estimating the causal effect Y₍₁₎−Y₍₀₎ is expressed by the following formula (1). Here, it is assumed that the outcome Y is predicted by a linear model, but the outcome Y may be predicted by a nonlinear model.

Y=α+β_{T}+β₁W₁+β₂W₂+β_{U}U (1)

In formula (1), Y is a value of an outcome, α is a constant term, β_{T}, β₁, β₂, β_{U} are partial regression coefficients, T is a value of a treatment judgment, W₁ and W₂ are values of observed confounding factors, and U is a value of an unobserved confounding factor. Furthermore, an outcome Y when T=1 corresponds to an outcome Y₍₁₎, and an outcome Y when T=0 corresponds to an outcome Y₍₀₎. Since the partial regression coefficient (3T affects a difference Y₍₁₎−Y₍₀₎ between Y₍₁₎ and Y₍₀₎, it is important to properly estimate β_{T} for estimating a causal effect.

However, since the value of the unobserved confounding factor U is unknown in the data set 200, the partial regression coefficient β_{U} representing a degree of influence of the unobserved confounding factor U on the outcome Y is not calculated. Accordingly, next, the following formula (2) excluding the term “+β_{U}U” in formula (1) is assumed.

Y=α+β_{T}T+β₁W₁+β₂W₂ (2)

Using formula (2), the medical information processing apparatus 1 can calculate each of the values of α, β_{T}, β₁, and β₂ by performing training by a multiple regression analysis, etc. based on the data set 200 for causal inference. However, since the term “+β_{U}U” is excluded, an influence of an uncalculated β_{U} value is added to each of the calculated α, β_{T}, β₁, and β₂ values. That is, since the calculated β_{T} value includes a bias, the medical information processing apparatus 1 cannot appropriately estimate the causal effect using formula (2).

Therefore, in the present embodiment, the medical information processing apparatus 1 estimates a causal effect by using a propensity score e, which is a probability that a patient will be assigned to a surgery (T=1). The propensity score e is a function of one or more observed confounding factors W, and ideally, if the propensity score e is appropriately estimated using all the confounding factors W and U, the causal effect is also appropriately estimated. As shown in FIG. 4, if it is assumed that the degree of influence of the unobserved confounding factor U on the doctor's judgment is unchanged before and after the CDS presentation, an amount of change ΔT in judgment from the first judgment T to the second judgment T′ is predicted from a value of an observed confounding factor W in the data set 200. The medical information processing apparatus 1 predicts the amount of change ΔT in judgment using a first function f that predicts a first propensity score T^{˜}, which is a predicted value of the first judgment T, and a second function g that predicts a second propensity score T′^{˜}, which is a predicted value of the second judgment T′. Here, the superscript tilde (^{˜}) indicates a predicted value, and indicates that the tilde is attached directly above the character. Further, since a value of a propensity score e of each patient is unknown at the time when the data set 200 is collected, a cell related to the propensity score e of each patient is indicated by “?”.

FIG. 6 is an example of a method for training parameters of a prediction function of a propensity score. First, before the CDS presentation, the first function f outputs the first propensity score T^{˜} with the observed confounding factors W₁ and W₂ as inputs. The first function f is modeled as in the following formula (3) using first parameters γ₁ and γ₂, which represent degrees of influence of the observed confounding factors on the doctor's judgment before the CDS presentation. Here, it is assumed that a propensity score is predicted by a linear model, but the propensity score may be predicted by a nonlinear model.

f(γ,W)=γ₁W₁+γ₂W₂=\tilde{T} (3)

In formula (3), f(γ, W) is the first function, γ₁ and γ₂ are the first parameters, W₁ and W₂ are values of the observed confounding factors, and T^{˜} is the first propensity score. Further, before the CDS presentation, a first prediction residual between a true value T of the first judgment and the first propensity score T^{˜} is expressed by “T−T^{˜} |²”.

Similarly, after the CDS presentation, the second function g outputs the second propensity score T′^{˜} with the observed confounding factors W₁ and W₂ as inputs. The second function g is modeled as in the following formula (4) using second parameters γ′₁ and γ′₂, which represent degrees of influence of the observed confounding factors on the doctor's judgment after the CDS presentation.

g(γ′,W)=γ₁′W₁+γ₂′W₂= [symbol] (4)

In formula (4), g(γ′, W) is the second function, γ′₁ and γ′₂ are second parameters, W₁ and W₂ are the values of the observed confounding factors, and T′^{˜} is the second propensity score. Further, after the CDS presentation, a second prediction residual between a true value T′ of the second judgment and the second propensity score T′^{˜} is expressed by “|T′−T′^{˜} |²”.

As described above, the medical information processing apparatus 1 models the first function f and the second function g that predict the true values T and T′ of the treatment judgment before and after the CDS presentation, respectively. A true value ΔT of the judgment change from before the CDS presentation to after the CDS presentation can be predicted from the observed confounding factors W under the assumption that the degree of influence of the unobserved confounding factor U is unchanged. That is, the true value ΔT of the judgment change in a difference before and after the CDS presentation can be predicted by using the first function f and the second function g.

In the difference before and after the CDS presentation, a third function h outputs a predicted value ΔT^{˜} of the judgment change with the observed confounding factors W₁ and W₂ as inputs. The third function h is modeled as in the following formula (5) using the first function f and the second function g.

$$\begin{matrix}h ( γ , {γ}^{′} , W ) = g ( {γ}^{′} , W ) - f ( γ , W ) \\ = ( {γ}_{1}^{′} - {γ}_{1} ) {W}_{1} + ( {γ}_{2}^{′} - {γ}_{2} ) {W}_{2} \\ = - \tilde{T} =\end{matrix} \qquad ( 5 )$$

In formula (5), h (γ, γ′, W) is the third function, and ΔT^{˜} is the predicted value of the judgment change. Further, in the difference before and after the CDS presentation, a third prediction residual between the true value ΔT of the judgment change and the predicted value ΔT^{˜} of the judgment change is expressed by “|ΔT−ΔT^{˜}|²”. In the present embodiment, the third function h is a difference obtained by subtracting the first function f from the second function g, but is not limited thereto. For example, the third function h may be the second function g divided by the first function f.

Using the first prediction residual, the second prediction residual, and the third prediction residual modeled as described above, the medical information processing apparatus 1 trains the parameters γ₁, γ₂, γ′₁, and γ′₂. At this time, a loss function L for training the parameters γ₁, γ₂, γ′₁, and γ′₂ is expressed by the following formula (6).

L(γ,γ′,W)=|T−\tilde{T}|²+|ΔT− [symbol] |²+|T′− [symbol] |² (6)

The medical information processing apparatus 1 trains each of the parameters γ₁, γ₂, γ′₁, and γ′₂ so as to minimize a value of the loss function L. The training at this time is specifically expressed by the following formula (7).

$${γ}_{1} , {γ}_{2} , {γ}_{1}^{′} , {γ}_{2}^{′} = \begin{matrix}arg min \\ {γ}_{1} , {γ}_{2} , {γ}_{1}^{′} , {γ}_{2}^{′}\end{matrix} \{ {\vert T - \tilde{T} \vert}^{2} + λ {\vert Δ T - \vert}^{2} + {\vert {T}^{′} - \vert}^{2} \} \qquad ( 7 )$$

In formula (7), λ is a hyperparameter. Specifically, the medical information processing apparatus 1 adjusts the hyperparameter λ so that the third prediction residual |ΔT−ΔT^{˜}|² does not become too much larger than the first prediction residual |T−T^{˜}|² and the second prediction residual |T′−T′^{˜}|². The medical information processing apparatus 1 may train the parameters γ₁, γ₂, γ′₁, and γ′₂ so as to minimize a sum of two terms including any one of the first prediction residual |T−T^{˜}|² and the second prediction residual |T′−T′^{˜}|², and the third prediction residual “|ΔT−ΔT^{˜}|²”.

As described above, the true value ΔT of the judgment change from before the CDS presentation to after the CDS presentation can be completely predicted only from the observed confounding factors W under the assumption that the degree of influence of the unobserved confounding factor U is unchanged. That is, in formula (6), the third prediction residual becomes 0, and only the degree of influence of the unobserved confounding factor U that is not explained by the observed confounding factors W in the first prediction residual and the second prediction residual remains as a residual. Therefore, the parameters γ₁, γ₂, γ′₁, and γ′₂ calculated by minimizing the above residual in formula (7) can be used for calculating the degree of influence of the unobserved confounding factor U from formula (6).

After the parameters γ₁, γ₂, γ′₁, and γ′₂ are trained, the medical information processing apparatus 1 calculates a degree of influence U′ of the unobserved confounding factor on the doctor's judgment T by the following formula (8) or (9).

U′=T−\tilde{T}=T−γ₁W₁−γ₂W₂ (8)

U′=T′− [symbol] =T′−γ₁′W₁−γ₂′W₂ (9)

As shown in formula (8) or (9), the medical information processing apparatus 1 calculates a difference by subtracting the predicted value of the judgment predicted using the trained parameters from the true value of the judgment before or after the CDS presentation, as the degree of influence of the unobserved confounding factor U on the doctor's judgment. It is assumed that the degree of influence U′ of the unobserved confounding factor on the doctor's judgment is smaller than the predicted degree of influence T^{˜} or T′^{˜} of the observed confounding factor.

Here, if it is assumed that there is a correlation between the degree of influence U′ of the unobserved confounding factor on the doctor's judgment and the degree of influence U of the unobserved confounding factor on the outcome, that is, that a ratio of a breakdown of the unobserved confounding factor U is unchanged, U′ is substituted for U. In this way, the medical information processing apparatus 1 estimates the outcome Y using the following formula (10).

Y=α+β_{T}T+β₁W₁+β₂W₂+β_{U}′U′ (10)

In formula (10), β′_{U} is a partial regression coefficient relating to the term including U′. Since the medical information processing apparatus 1 predicts the outcome Y based on the data set 200 using the U′ estimated as described above, the partial regression coefficient β_{T} is not biased. Therefore, the medical information processing apparatus 1 can appropriately estimate the causal effect based on formula (10). When the CDS model 3 presents support information based on formula (2), which does not consider the degree of influence of the unobserved confounding factor U at the time of collecting the data set 200, the medical information processing apparatus 1 may update the CDS model 3 so as to present support information based on formula (10), which does consider the degree of influence U of the unobserved confounding factor.

For predication of the outcome Y, an existing method (doubly robust estimation, X-learner, R-learner, DR-learner, etc.) that combines a propensity score and outcome prediction may be used. Subsequently, the medical information processing apparatus 1 may calculate various causal effects (an average treatment effect (ATE), a conditional average treatment effect (CATE), an individual treatment effect (ITE), etc.) using the predicted outcome Y.

Further, the medical information processing apparatus 1 or the CDS model 3 may output support information based on a predicted causal effect. For example, if a predicted causal effect Y₍₁₎−Y₍₀₎ has a positive sign, the medical information processing apparatus 1 may output a recommended treatment corresponding to an intervention T (i.e., T=1) that produces an outcome Y₍₁₎ as the support information. Conversely, if the causal effect Y₍₁₎−Y₍₀₎ has a negative sign, the medical information processing apparatus 1 may output a recommended treatment corresponding to an intervention T (i.e., T=0) that produces an outcome. Y₍₀₎ as the support information. Further, the medical information processing apparatus 1 or the CDS model 3 may output a ratio of a degree of influence of each confounding factor in support information.

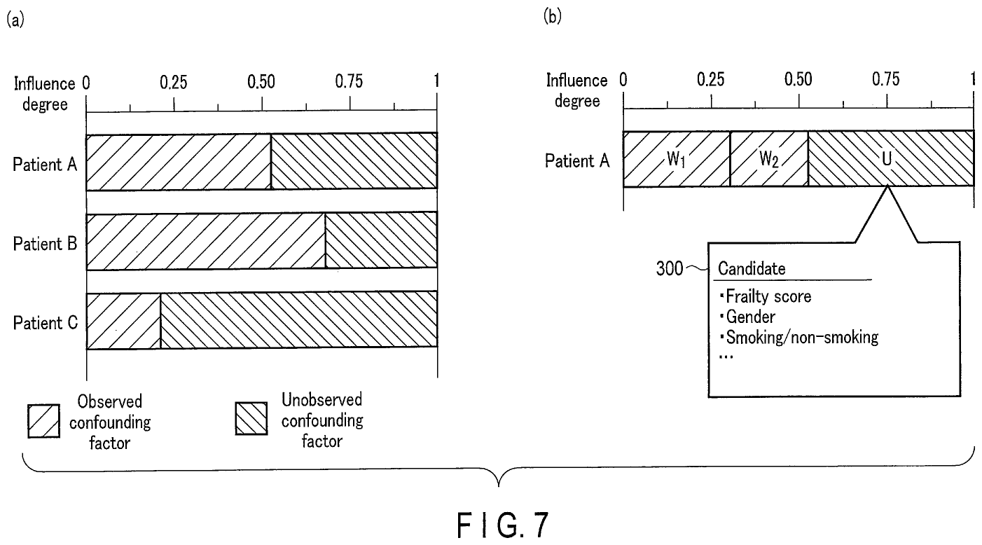

FIG. 7 is an example of a degree of influence of each confounding factor on support information. FIGS. 7(a) and 7(b) can be displayed on the display 13 of the medical information processing apparatus 1.

In FIG. 7(a), a degree of influence of each confounding factor on each piece of support information presented by the medical information processing apparatus 1 for each patient (patient A, patient B, and patient C) is shown by a bar graph. Specifically, the degree of influence of each confounding factor corresponds to a ratio of each value obtained by standardizing the partial regression coefficients β₁, β₂, and β′_{U} in formula (10) to a sum of the values of the standardized partial regression coefficients β₁, β₂, and β′_{U}. For example, the value of the standardized β′_{U} in the sum of the standardized partial regression coefficients β₁, β₂, and vu corresponds to the degree of influence of the unobserved confounding factor U. The degree of influence of each original confounding factor before standardization is unchanged.

For example, the degree of influence of the observed confounding factor W on the support information presented to the patient A is “0.55”, and the degree of influence of the unobserved confounding factor U is “0.45”. Similarly, the degree of influence of the observed confounding factor W on the support information presented to the patient B is “0.70”, and the degree of influence of the unobserved confounding factor U is “0.30”. By referring to FIG. 7(a) displayed on the display 13, the user who uses the medical information processing apparatus 1 can check a ratio of a degree of influence of each confounding factor in support information output in consideration of a degree of influence of an unobserved confounding factor.

While FIG. 7(a) is displayed, the user who uses the medical information processing apparatus 1 can select a bar graph related to a desired patient by operating the input interface 14. For example, when the bar graph related to the patient A is selected, the screen shifts from FIG. 7(a) to a display screen of FIG. 7(b).

In FIG. 7(b), both the degree of influence of the observed confounding factor W and the degree of influence of the unobserved confounding factor U are calculated, and a breakdown of the bar graph is displayed. Here, by analyzing predetermined data, the medical information processing apparatus 1 may display one or more candidates for the unobserved confounding factor U in a window 300. Specifically, the window 300 displays “frailty score”, “gender”, “smoking/non-smoking”, etc. as a plurality of candidates for the unobserved confounding factor. As a method for determining a candidate for the unobserved confounding factor, for example, a user (data scientist or knowledge providing doctor) who performs and supports the data analysis may manually select the candidate. Alternatively, for example, the medical information processing apparatus 1 may determine, among observed confounding factors used in other data processing, a confounding factor not selected as an observed confounding factor in a processing result of the medical information processing apparatus 1 as a candidate for the unobserved confounding factor U.

In order to present the candidate for the unobserved confounding factor U, for example, the medical information processing apparatus 1 puts one or more unobserved confounding factors U into the CDS model 3 as a part of the confounding factors W, and calculates a degree of influence again using the same method. If the degree of influence of the unobserved confounding factor U decreases by a certain amount or more before and after the processing, the medical information processing apparatus 1 may present the factor put in the CDS model 3 as the above candidate. The above processing is premised on the presence of an unobserved confounding factor U that is obtained as data but is not recognized as an observed confounding factor W.

Above are descriptions of the medical information processing apparatus 1 according to the embodiment. The medical information processing apparatus 1 indirectly quantifies a degree of influence of an unobserved confounding factor based on a degree of influence of an observed confounding factor. According to the medical information processing apparatus 1, it is possible to quantify a degree of influence of an unobserved confounding factor that affects a doctor's judgment. As a result, the doctor can quantitatively assess a degree of reliability of causal inference. That is, the medical information processing apparatus 1 can improve the reliability of causal inference.

Here, a case in which the doctor makes a judgment by considering only an observed confounding factor is assumed. Similarly, in this case, the medical information processing apparatus 1 acquires a first numerical value corresponding to the doctor's judgment before presentation of support information (CDS) and a second numerical value corresponding to the doctor's judgment after the presentation of the support information (CDS). Subsequently, the medical information processing apparatus 1 calculates a first propensity score, which is a predicted value of the first numerical value, and a second propensity score, which is a predicted value of the second numerical value, based on the observed confounding factor. Finally, the medical information processing apparatus 1 calculates a difference between the first numerical value and the first propensity score, or a difference between the second numerical value and the second propensity score, as a degree of influence of an unobserved confounding factor. Therefore, if the doctor makes a judgment by considering only the observed confounding factor, the degree of influence of the unobserved confounding factor is calculated as “0”. Thereby, the user who uses the medical information processing apparatus 1 can confirm that the influence of the unobserved confounding factor is not included in that doctor's judgment.

According to at least one embodiment described above, causal inference can be appropriately performed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information processing apparatus comprising processing circuitry configured to:
acquire a first numerical value corresponding to a result judged by a user based on an observed confounding factor or based on the observed confounding factor and an unobserved confounding factor;
acquire a second numerical value corresponding to a result judged by the user based on the observed confounding factor and first support information that supports a judgment of the user or based on the observed confounding factor, the unobserved confounding factor, and the first support information;
extract a first difference between the first numerical value and the second numerical value; and
calculate a degree of influence of the unobserved confounding factor on the judgment of the user based on the first difference and the observed confounding factor.

2. The medical information processing apparatus according to claim 1, wherein the processing circuitry is configured to:
extract a second difference between a first propensity score and a second propensity score, the first propensity score being output from a first function with the observed confounding factor as an input and being a predicted value of the first numerical value, the second propensity score being output from a second function with the observed confounding factor as an input and being a predicted value of the second numerical value;
train a first parameter of the first function and a second parameter of the second function so as to minimize a prediction residual between the first difference and the second difference; and
calculate a difference between the first numerical value and the first propensity score predicted by using the trained first parameter, or a difference between the second numerical value and the second propensity score predicted by using the trained second parameter, as the degree of influence of the unobserved confounding factor.

3. The medical information processing apparatus according to claim 1, wherein
the processing circuitry is configured to update a model that outputs the first support information by using the degree of influence of the unobserved confounding factor.

4. The medical information processing apparatus according to claim 1, wherein
the processing circuitry is configured to estimate a causal effect of the judgment of the user on an outcome based on the degree of influence of the unobserved confounding factor.

5. The medical information processing apparatus according to claim 4, wherein
the processing circuitry is configured to output second support information that supports the judgment of the user based on the causal effect.

6. The medical information processing apparatus according to claim 5, wherein
the processing circuitry is configured to output a ratio of the degree of influence of the unobserved confounding factor in the second support information.

7. The medical information processing apparatus according to claim 5, wherein
the processing circuitry is configured to output a candidate for the unobserved confounding factor that affects the second support information.

8. A medical information processing system comprising a medical care information database and a medical information processing apparatus, wherein
the medical care information database stores a first numerical value corresponding to a result judged by a user based on an observed confounding factor or based on the observed confounding factor and an unobserved confounding factor, and a second numerical value corresponding to a result judged by the user based on the observed confounding factor and first support information that supports a judgment of the user or based on the observed confounding factor, the unobserved confounding factor, and the first support information, and
the medical information processing apparatus includes processing circuitry configured to:
acquire the first numerical value and the second numerical value;
extract a first difference between the first numerical value and the second numerical value; and
calculate a degree of influence of the unobserved confounding factor on the judgment of the user based on the first difference and the observed confounding factor.
